(19) United States
(12) Patent Application Publication
(10) Pub. No.: US 2022/0167650 A1
(43) Pub. Date: Jun. 2, 2022

(54) HIGH-INTENSITY OUTDOOR SPORTS FOOD SUPPLEMENT RICH IN BIOLOGICAL IONS AND PREPARATION METHOD THEREOF

(71) Applicant: Qufu Normal University, Jining (CN)

(72) Inventors: Ge YANG, Jining (CN); Ruixia GAO, Jining (CN); Chengchuan CHE, Jining (CN); Jinfeng LIU, Jining (CN); Zhijin GONG, Jining (CN)

(21) Appl. No.: 17/369,441

(22) Filed: Jul. 7, 2021

(30) Foreign Application Priority Data
Nov. 27, 2020 (CN) 202011362705.X

(51) Int. Cl. A23L 33/105; A23L 33/15; A23L 33/175; A23L 33/16; A61K 36/79; A61K 45/06; A61K 31/198

(52) U.S. Cl. CPC A23L 33/105; A23L 33/15; A23L 33/175; A61K 31/198; A61K 36/79; A61K 45/06; A23L 33/16

(57) ABSTRACT

The present disclosure relates to the technical field of food supplement application, in particular to a high-intensity outdoor sports food supplement rich in biological ions and a preparation method thereof. The sports food supplement is prepared using the following components: poly-γ-glutamic acid, magnesium sulfate, sodium carbonate, ammonium bicarbonate, potassium magnesium aspartate, potassium chloride, sodium citrate, potassium citrate, hydroxide calcium, zinc sulfate, antioxidant, preservative, Schisandra chinensis extraction solution, and water. The present disclosure promotes faster recovery of middle-aged men during exercise, reduces the risk of injury, and improves the ability of high-intensity running. The concentrated solution provided by the present disclosure has no bitter taste, a slightly sweet and refreshing taste, good palatability and good stability. The present disclosure is simple in preparation, and has a wide application range, which not only can be applied to sports drinks, but also can be added to sports food.

### CROSS REFERENCE TO RELATED APPLICATION(S)

This patent application claims the benefit and priority of Chinese Patent Application No. 202011362705.X, filed on Nov. 27, 2020, the disclosure of which is incorporated by reference herein in its entirety as part of the present application.

### TECHNICAL FIELD

The present disclosure relates to the technical field of food supplement application, in particular to a high-intensity outdoor sports food supplement rich in biological ions and a preparation method thereof.

### BACKGROUND ART

As the saying goes, “When a person passes the age of 40, it is like that the sun has passed the highest point in the sky”, which means that the human body has already begun to decline in terms of flexibility, speed, endurance and strength. In particular, the basal metabolic rate of middle-aged men is only about 90% of that at the age of 25. The body does not work as well as it used to be due to intensified wear and tear on the joints, gradual decrease and reduction of the muscle strength, and the decline of bone density. Therefore, it is necessary to maintain appropriate workouts, but middle-aged men are prone to slow recovery after exercise, easy injury, and low exercise intensity.

Exercise induces energy depletion (consumption of glycogen, ATP, creatine phosphate), muscle acidosis, accumulation of metabolites (lactic acid, ADP, phosphoric acid, ammonia, inosine monophosphate, etc.), excitability of the central nervous system, spinal cord, neuromuscular junctions and muscle membranes, oxidative stress. Fast and effective recovery from fatigue not only helps improve the body's athletic ability, but also helps enhance physical health and self-confidence.

Due to exercise, a large amount of electrolytes such as sodium, potassium, chlorine, and magnesium are lost along with sweating. For those who exercise for a long time and with high intensity, they should consider supplementing electrolytes to restore their physical ability. So, in order to adapt to high-volume and high-intensity training, a balanced diet alone is not sufficient to provide the large amount of material consumed during training, and it is necessary to use functional sports food supplements to help improve endurance and restore physical strength and exercise capacity, and therefore, help withstand higher training pressure and adapt to greater additional training stress, promote faster recovery during exercise and reduce the risk of injury, and improve high-intensity exercise capacity.

Poly-γ-glutamic acid, abbreviated as γ-PGA, is a special anionic natural polymer, which is an isomorphic polyamide formed by bonding an α-amino group and a γ-carboxyl group via an amide bond. As a kind of extracellular polymer synthesized by microorganisms, γ-PGA is a multifunctional, environment-friendly biodegradable polymer material, which typically has a molecular weight of about 10 to 1000 kDa. Due to its unique physical, chemical and biological properties, such as good water solubility and super adsorption, γ-PGA can be completely degraded both biologically and naturally. So, it is non-toxic and harmless to the environment, and edible. When added to a food product, it allows the food product to have increased calcium and mineral absorption, less debris, anti-oxidation, resistance to soften, maintained flavor, good taste, extended shelf life, etc., so it has been widely used in the field of food.

Magnesium is indispensable for the synthesis of ATP in the human body. Studies have found that magnesium is mainly involved in muscle contraction, while helping to improve the body's sugar tolerance, effectively enhancing physical power and strength. Every day, magnesium plays an important role in approximately 600 chemical reactions in the human body. In fact, magnesium is very important for the production of energy, the regulation of nervous system, the maintenance of genes, and the formation of proteins. Calcium can promote the activity of certain enzymes in the body, regulate the activity of enzymes, participate in the activities of nerves and muscles as well as the release of neurotransmitters, and modulate the secretion of hormones. Calcium is also needed for blood coagulation, cell adhesion, and muscle contraction activities. Calcium also has the functions of regulating heart rhythm, reducing cardiovascular permeability, controlling inflammation and edema, and maintaining acid-base balance. A specific ratio maintained between calcium and other ions such as magnesium, potassium, and sodium keeps the nerves and muscles in a normal response. Calcium can regulate heartbeats, keep the heart to contract and relax continuously and alternately, and maintain the contraction of muscles and the transmission of nerve impulses. The production of more than 200 kinds of enzymes in the human body and the nine chemical signal transmission systems in the brain-neurotransmitting substances are inseparable from zinc. That is because zinc plays an important role in maintaining cell integrity, cell proliferation, gene regulation, nucleic acid metabolism and immune function. Zinc also helps maintain the function of the brain. Zinc deficiency will hinder protein synthesis in the body. Moreover, the production of male testosterone and growth hormone is inseparable from the support of zinc. Potassium ions are the main cations in the intracellular fluid, and 98% of the potassium in the body is present in the cells. Both myocardium and neuromuscular muscles need a relatively constant potassium ion concentration to maintain normal stress. At present, there are few reports about high-intensity outdoor sports food supplements for middle-aged men.

### SUMMARY

It is an object of the present disclosure to provide a high-intensity outdoor sports food supplement rich in biological ions, which promotes faster recovery of middle-aged men during exercise, reduces the risk of injury, and improves the ability of high-intensity running.

The present disclosure also provides a method for the preparation of a high-intensity outdoor sports food supplement rich in biological ions.

The technical solutions used in the present disclosure to achieve the above objects are as follows:

The present disclosure provides a high-intensity outdoor sports food supplement rich in biological ions, which is prepared using the following components: 10-15 parts of poly-γ-glutamic acid, 6-15 parts of magnesium sulfate, 3-10 parts of sodium carbonate, 4-12 parts of ammonium bicarbonate, 5-20 parts of potassium magnesium aspartate, 2-10 parts of potassium chloride, 5-10 parts of sodium citrate, 3-12 parts of potassium citrate, 10-15 parts of hydroxide calcium, 5-20 parts of zinc sulfate, 1 part of antioxidant, 0.05 part of preservative, 3-5 parts of Schisandra chinensis extraction solution, and 20 parts of water.

Further, the potassium magnesium aspartate is an equal mixture of potassium L-aspartate and magnesium L-aspartate; the antioxidant is vitamin C, vitamin E, citric acid or a combination thereof; the preservative is potassium sorbate or sodium benzoate.

The present disclosure also provides a method for the preparation of a high-intensity outdoor sports food supplement rich in biological ions, comprising the following steps:

(1) Mixing magnesium sulfate, sodium carbonate, ammonium bicarbonate, potassium magnesium aspartate, potassium chloride, sodium citrate, potassium citrate, zinc sulfate, antioxidant, preservative, and water, and heating to 90-120° C. with stirring to obtain a mixed solution;

(2) Allowing the above mixed solution to stand, then cooling to room temperature, and filtering out solid residues to obtain a mixed clear solution;

(3) Mixing poly-γ-glutamic acid with a Schisandra chinensis extraction solution and calcium hydroxide uniformly, heating the mixture to 40° C. in a water bath, stirring for 5 minutes, and cooling to room temperature to obtain a poly-γ-glutamic acid mixture;

(4) Mixing the mixed clear solution and the poly-γ-glutamic acid mixture uniformly, and concentrating to obtain a concentrated solution as a sports food supplement.

Further, in step (2), the standing time is 20-30 hours.

The concentrated solution prepared by the present disclosure has a density of 1.12.

The Schisandra chinensis extraction solution used in the present disclosure is prepared by the following preparation method: Dried Schisandra chinensis is mashed; then 1-2% of amylase by weight of the Schisandra chinensis, 0.2% of polyglycerol fatty acid ester, and 5-fold by weight of water are added to obtain a mixture; the mixture is heated to 38-42° C., kept warm for 1.5 hours, and then heated to 90° C. to inactivate the enzyme for 1 minute; after cooling to room temperature, the mixture is concentrated to be one-fifth of the original volume.

The poly-γ-glutamic acid used in the present disclosure is obtained by fermentation and extraction from Bacillus licheniformis. The fermentation extraction method includes the following steps:

a. A basal medium is weighed and dissolved to obtain a basal medium solution, then the components of an optimized medium are weighed and added to the basal medium solution to set a constant volume with the pH being adjusted to 7.5 with solid NaOH; b. The medium is divided and placed into beaker flasks (50 ml per flask), and sterilized at 100-150° C.; Bacillus licheniformis is inoculated into the medium at an inoculum of 2-10%, and cultured at 30-50° C. on a shaker for 3-7 days to obtain a fermentation broth;

b. The fermentation broth is centrifuged; 2- to 7-fold by volume of ethanol is added to the supernatant to precipitate for 10-24 hours; centrifugation is performed; and then the obtained supernatant is precipitated again with 2- to 6-fold by volume of ethanol; centrifugation is performed; and the precipitate is dried to a constant weight to obtain a constant-weight sample;

c. The constant-weight sample is dissolved in distilled water, and purified by dialysis to obtain a purified sample;

d. The purified sample is dissolved in distilled water, and spray-dried to obtain poly-γ-glutamic acid powder.

In the above preparation process of poly-γglutamic acid, in step (a), the volume ratio of the optimized medium to the basal medium is 1:4.

The basal medium used in the present disclosure consists of the following raw materials: tryptone 10 g/L, yeast extract 5 g/L, and NaCl 10 g/L; the optimized medium consists of the following raw materials: NaCl 10-15 g/L, α-ketoglutaric acid 1.0-2.5 g/L, Mn(II) 0.02-0.1 g/L, L-glutamine 0.2-1.0 g/L, and glycerol 5-15 g/L.

The beneficial effects of the present disclosure include:

(1) The present disclosure promotes faster recovery of middle-aged men during exercise, reduces the risk of injury, and improves the ability of high-intensity running.

(2) The concentrated solution provided by the present disclosure has no bitter taste, a slightly sweet and refreshing taste, good palatability and good stability.

(3) The concentrated solution provided by the present disclosure is simple in preparation, and has a wide application range, which not only can be applied to sports drinks, but also can be added to sports food.

### DETAILED DESCRIPTION OF THE EMBODIMENTS

The specific embodiments of the present disclosure are illustrated by the following examples, but the content, protection of which is asked for in the present disclosure, is not limited thereto.

### Example 1

1) 15 g of magnesium sulfate, 10 g of sodium carbonate, 12 g of ammonium bicarbonate, 20 g of potassium magnesium aspartate, 10 g of potassium chloride, 10 g of sodium citrate, 12 g of potassium citrate, 20 g of zinc sulfate, 1 g of antioxidant, 0.05 g of preservative, and 20 g of water are mixed, and heated to 120° C. with stirring to obtain a mixed solution;

2) The above mixed solution is allowed to stand for 24 hours, then cooled to room temperature, and filtered out solid residues to obtain a mixed clear solution;

3) 15 g of poly-γ-glutamic acid is mixed with 3 g of Schisandra chinensis extraction solution and 15 g of calcium hydroxide uniformly to obtain a poly-γ-glutamic acid mixture;

4) The mixed clear solution is mixed with the poly-γ-glutamic acid mixture uniformly, and concentrated to obtain a concentrated solution as a sports food supplement.

### Example 2

1) 12 g of magnesium sulfate, 5 g of sodium carbonate, 6 g of ammonium bicarbonate, 7 g of potassium magnesium aspartate, 8 g of potassium chloride, 10 g of sodium citrate, 9 g of potassium citrate, 17 g of zinc sulfate, 1 g of antioxidant, 0.05 g of preservative, and 20 g of water are mixed, and heated to 100° C. with stirring to obtain a mixed solution;

2) The above mixed solution is allowed to stand for 24 hours, then cooled to room temperature, and filtered out solid residues to obtain a mixed clear solution;

3) 10 g of poly-γ-glutamic acid is mixed with 3 g of Schisandra chinensis extraction solution and 13 g of calcium hydroxide uniformly to obtain a poly-γ-glutamic acid mixture;

4) The mixed clear solution is mixed with the poly-γ-glutamic acid mixture uniformly, and concentrated to obtain a concentrated solution as a sports food supplement.

### Example 3

1) 6 g of magnesium sulfate, 3 g of sodium carbonate, 4 g of ammonium bicarbonate, 5 g of potassium magnesium aspartate, 2 g of potassium chloride, 5 g of sodium citrate, 3 g of potassium citrate, 5 g of zinc sulfate, 1 g of antioxidant, 0.05 g of preservative, and 20 g of water are mixed, and heated to 90° C. with stirring to obtain a mixed solution;

2) The above mixed solution is allowed to stand for 24 hours, then cooled to room temperature, and filtered out solid residues to obtain a mixed clear solution;

3) 15 g of poly-γ-glutamic acid is mixed with 5 g of Schisandra chinensis extraction solution and 10 g of calcium hydroxide uniformly to obtain a poly-γ-glutamic acid mixture;

4) The mixed clear solution is mixed with the poly-γ-glutamic acid mixture uniformly, and concentrated to obtain a concentrated solution as a sports food supplement.

### Comparative Example 1

1) 15 g of magnesium sulfate, 10 g of sodium carbonate, 12 g of ammonium bicarbonate, 20 g of potassium magnesium aspartate, 10 g of potassium chloride, 10 g of sodium citrate, 12 g of potassium citrate, 20 g of zinc sulfate, 1 g of antioxidant, 0.05 g of preservative, and 20 g of water are mixed, and heated to 120° C. with stirring to obtain a mixed solution;

2) The above mixed solution is allowed to stand for 24 hours, then cooled to room temperature, and filtered out solid residues to obtain a mixed clear solution;

3) The mixed clear solution is mixed with 15 g of poly-γ-glutamic acid uniformly, and concentrated to obtain a concentrated solution as a sports food supplement.

### Comparative Example 2

1) 15 g of magnesium sulfate, 10 g of sodium carbonate, 12 g of ammonium bicarbonate, 20 g of potassium magnesium aspartate, 10 g of potassium chloride, 10 g of sodium citrate, 12 g of potassium citrate, 20 g of zinc sulfate, 1 g of antioxidant, 0.05 g of preservative, and 20 g of water are mixed, and heated to 120° C. with stirring to obtain a mixed solution;

2) The above mixed solution is allowed to stand for 24 hours, then cooled to room temperature, and filtered out solid residues to obtain a mixed clear solution;

3) 15 g of poly-γ-glutamic acid is mixed with 3 g of glycyrrhizin and 15 g of calcium hydroxide uniformly to obtain a poly-γ-glutamic acid mixture;

4) The mixed clear solution is mixed with the poly-γ-glutamic acid mixture uniformly, and concentrated to obtain a concentrated solution as a sports food supplement.

Effect Example 1: The influence of concentrated solution of biological ions of the present disclosure on the recovery from exercise fatigue, injury risk and running ability of mice1.

1. Experimental animals: 4-week-old male Kunming mice weighing 20.0-22.0 grams. The indoor temperature and relative humidity of the animal experiment center are 23±1° C. and 60±1%, respectively, and the lighting is set such that it is 12 hours for daytime (6:00 to 18:00) and 12 hours for night. All mice are bred adaptively for 1 week, wherein the mice with dysplasia are excluded, and the remaining mice are randomly divided into groups, including blank group, Example 1 group, Example 2 group, Example 3 group, Comparative Example 1 group, and Comparative Example 2 groups. The number of mice in each group is 10.

2. Test substances: The concentrated solutions of Examples 1-3 and Comparative Examples 1-2 are mixed with purified water at a ratio of 1:9 to obtain test substances 1-4, wherein the test substance of the blank group is purified water.

3. Experimental Method

After one week of adaptive feeding, the mice are tied with a lead wire at the tail, which is 10% of the body weight of the mouse, to carry out the weight-bearing running experiment of mice. The treadmill is a wheel treadmill, and the treadmill speed is set to be within the range of 1-60 m/min, in a constant speed mode. The timer is started when a mouse is gently put into the treadmill. When the mouse stops running for 10 seconds, the timer is stopped. The time of weight-bearing running of the mouse is calculated and recorded it as the initial running time. During the test, if the mouse sits and rests, a glass rod is immediately used to stimulate it to run.

Thirty minutes after the end of the weight-bearing running experiment, the corresponding test substance is given to the mice in each group by gavage, wherein the gavage amount is 0.2 mL/10 g body weight, and the same amount of the test substance is given again by gavage at time 60 minutes and 90 minutes, respectively. After the completion of gavage at time 90 minutes, the mice in each group are randomly divided into two groups, that is, blank-1 group, blank-2 group, Example 1-1 group, Example 1-2 group, Example 2-1 group, Example 2-2 group, Example 3-1 group, Example 3-2 group, Comparative Example 1-1 group, Comparative Example 1-2 group, Comparative Example 2-1 group, and Comparative Example 2-2 group.

At time 120 minutes after the end of the weight-bearing running experiment, the mice in blank-1 group, Example 1-1 group, Example 2-1 group, Example 3-1 group, Comparative Example 1-1, Comparative Example 2-1 group are subjected to the weight-bearing running experiment again, and the weight-bearing running time of the mice is recorded as the running time at time 2 hours after exhaustion. The experimental conditions are the same as before. The 2-hour recovery rate is calculated, wherein the 2-hour recovery rate=the running time at time 2 hours after exhaustion/the initial running time.

At time 240 minutes after the end of the weight-bearing running experiment, the mice in blank-2 group, Example 1-2 group, Example 2-2 group, Example 3-2 group, Comparative Example 1-2, Comparative Example 2-2 group are subjected to the weight-bearing running experiment again, and the weight-bearing running time of the mice is recorded as the running time at time 4 hours after exhaustion. The experimental conditions are the same as before, and the 4-hour recovery rate is calculated, wherein the 4-hour recovery rate=the running time at time 4 hours after exhaustion/the initial running time. The specific experimental results are shown in Table 1.

TABLE 1
The influence of test substances on the weight-bearing running time of
mice

| | | | Running | Recovery | Running | Recovery |
|---|---|---|---|---|---|---|
| | | | time at | rate at | time at | rate at |
| | Body weight | Initial running | time 2 | time 2 | time 4 | time 4 |
| | (g) | time (s) | hours (s) | hours | hours (s) | hours |
| Blank group | 28.7 | 332 | 124 | 37.35% | 156 | 46.99% |
| Ex. 1 group | 28.5 | 329 | 231 | 70.21% | 272 | 82.67% |
| Ex. 2group | 29.4 | 338 | 169 | 50% | 183 | 54.14% |
| Ex. 3 group | 28.3 | 324 | 142 | 43.83% | 166 | 51.23% |
| Comp. Ex. 1 group | 28.9 | 326 | 103 | 31.59% | 157 | 48.16% |
| Comp. Ex. 1 group | 29.3 | 328 | 110 | 33.54% | 162 | 49.39% |

The experimental results in Table 1 show that the weight-bearing running time of mice at time 2 hours and 4 hours after exhaustion is shorter than the initial running time, but the recovery rate of mice in Examples 2-3 groups at time 2 hours and 4 hours after exhaustion is higher than the blank group, indicating that the test substances in Examples 2-3 groups exhibit an effect of promoting exercise recovery. And the recovery rate of mice in Example 1 group at time 2 hours after exhaustion is 70.21%, which is significantly higher than the blank group and Examples 2-3 groups, and even higher than the recovery rate of mice in the blank group and Examples 2-3 groups at time 4 hours after exhaustion. The recovery rate of mice in Example 1 group at time 4 hours after exhaustion has reached 82.67%, which is significantly higher than the blank group and Example 2-4 groups. Therefore, the experimental results in Table 1 prove that the concentrated solution of biological ions prepared by the present disclosure has a significantly better effect of promoting exercise fatigue recovery, and the recovery time is also significantly shorter, and the running ability is enhanced, thus indicating that the concentrated solution of biological ions of the present disclosure has achieved a rapid and efficient synergistic effect of promoting exercise fatigue recovery and enhancing running ability through a reasonable formula.

The above are preferred embodiments of the present disclosure, but they are not intended to limit the present disclosure. Those skilled in the art can make improvements and changes to the embodiments disclosed herein without departing from the scope and spirit of the present disclosure.

1. A high-intensity outdoor sports food supplement rich in biological ions, wherein it is prepared using the following components: 10-15 parts of poly-γ-glutamic acid, 6-15 parts of magnesium sulfate, 3-10 parts of sodium carbonate, 4-12 parts of ammonium bicarbonate, 5-20 parts of potassium magnesium aspartate, 2-10 parts of potassium chloride, 5-10 parts of sodium citrate, 3-12 parts of potassium citrate, 10-15 parts of hydroxide calcium, 5-20 parts of zinc sulfate, 1 part of antioxidant, 0.05 part of preservative, 3-5 parts of Schisandra chinensis extraction solution, and 20 parts of water.

2. The high-intensity outdoor sports food supplement rich in biological ions according to claim 1, wherein the potassium magnesium aspartate is an equal mixture of potassium L-aspartate and magnesium L-aspartate; the antioxidant is vitamin C, vitamin E, citric acid or a combination thereof; the preservative is potassium sorbate or sodium benzoate.

3. A method for the preparation of a high-intensity outdoor sports food supplement rich in biological ions, the high-intensity outdoor sports food supplement rich in biological ions, wherein it is prepared using the following components: 10-15 parts of poly-γ-glutamic acid, 6-15 parts of magnesium sulfate, 3-10 parts of sodium carbonate, 4-12 parts of ammonium bicarbonate, 5-20 parts of potassium magnesium aspartate, 2-10 parts of potassium chloride, 5-10 parts of sodium citrate, 3-12 parts of potassium citrate, 10-15 parts of hydroxide calcium, 5-20 parts of zinc sulfate, 1 part of antioxidant, 0.05 part of preservative, 3-5 parts of Schisandra chinensis extraction solution, and 20 parts of water, comprising the following steps:
(1) Mixing magnesium sulfate, sodium carbonate, ammonium bicarbonate, potassium magnesium aspartate, potassium chloride, sodium citrate, potassium citrate, zinc sulfate, antioxidant, preservative, and water, and heating to 90-120° C. with stirring to obtain a mixed solution;
(2) Allowing the above mixed solution to stand, then cooling to room temperature, and filtering out solid residues to obtain a mixed clear solution;
(3) Mixing poly-γ-glutamic acid with a Schisandra chinensis extraction solution and calcium hydroxide uniformly, heating the mixture to 40° C. in a water bath, stirring for 5 minutes, and cooling to room temperature to obtain a poly-γ-glutamic acid mixture;
(4) Mixing the mixed clear solution and the poly-γ-glutamic acid mixture uniformly, and concentrating to obtain a concentrated solution as a sports food supplement.

4. The method for preparation according to claim 3, wherein the standing time in step (2) is 20-30 hours.

5. The method for preparation according to claim 3, wherein the concentrated solution in step (4) has a density of 1.12

6. The method for preparation according to claim 4, wherein the concentrated solution in step (4) has a density of 1.12.

7. A method for the preparation of a high-intensity outdoor sports food supplement rich in biological ions, the high-intensity outdoor sports food supplement rich in biological ions, wherein it is prepared using the following components: 10-15 parts of poly-γ-glutamic acid, 6-15 parts of magnesium sulfate, 3-10 parts of sodium carbonate, 4-12 parts of ammonium bicarbonate, 5-20 parts of potassium magnesium aspartate, 2-10 parts of potassium chloride, 5-10 parts of sodium citrate, 3-12 parts of potassium citrate, 10-15 parts of hydroxide calcium, 5-20 parts of zinc sulfate, 1 part of antioxidant, 0.05 part of preservative, 3-5 parts of Schisandra chinensis extraction solution, and 20 parts of water, wherein the potassium magnesium aspartate is an equal mixture of potassium L-aspartate and magnesium L-aspartate; the antioxidant is vitamin C, vitamin E, citric acid or a combination thereof; the preservative is potassium sorbate or sodium benzoate, comprising the following steps:
(1) Mixing magnesium sulfate, sodium carbonate, ammonium bicarbonate, potassium magnesium aspartate, potassium chloride, sodium citrate, potassium citrate, zinc sulfate, antioxidant, preservative, and water, and heating to 90-120° C. with stirring to obtain a mixed solution;
(2) Allowing the above mixed solution to stand, then cooling to room temperature, and filtering out solid residues to obtain a mixed clear solution;
(3) Mixing poly-γ-glutamic acid with a Schisandra chinensis extraction solution and calcium hydroxide uniformly, heating the mixture to 40° C. in a water bath, stirring for 5 minutes, and cooling to room temperature to obtain a poly-γ-glutamic acid mixture;
(4) Mixing the mixed clear solution and the poly-γ-glutamic acid mixture uniformly, and concentrating to obtain a concentrated solution as a sports food supplement.

8. The method for preparation according to claim 7, wherein the standing time in step (2) is 20-30 hours.

9. The method for preparation according to claim 7, wherein the concentrated solution in step (4) has a density of 1.12.

10. The method for preparation according to claim 8, wherein the concentrated solution in step (4) has a density of 1.12.
